(19) United States
(12) Patent Application Publication
(10) Pub. No.: US 2020/0175389 A1
(43) Pub. Date: Jun. 4, 2020

(54) MEDICAL PROCEDURE ASSESSMENT AND EVALUATION

(71) Applicant: International Business Machines Corporation, Armonk, NY (US)

(72) Inventors: Marcia Ito, Sao Paulo (BR); Joel Luís Carbonera, Porto Alegre (BR); Ricardo Luis Ohta, Sao Paulo (BR)

(21) Appl. No.: 16/203,724

(22) Filed: Nov. 29, 2018

(51) Int. Cl. G06N 5/04; G06N 7/00; G06N 20/00; G16H 50/20; G16H 70/20

(52) U.S. Cl. CPC G06N 5/04; G06N 7/005; G16H 70/20; G16H 50/20; G06N 20/00

(57) ABSTRACT

A computer-implemented method for medical procedure assessment and evaluation is provided. The method receives, by a processor, a claim record and stores the claim record as a claim. The method classifies, by the processor, the claim using an intelligent system including an inference engine and knowledge base and classifies, by the processor, a probability of a diagnostic using the intelligent system. The method calculates, by the processor, using the probability of the diagnostic and the claim a probability of consistency of the diagnostic using the intelligent system and presents, by the processor, the probability of consistency of the diagnostic to a user.

### BACKGROUND

The present invention generally relates to health care, and more specifically, to a system and method for medical procedure and assessment.

Health care is a growing field with ever more resources devoted to the medical care of a population of a country. Thus, it is important to use limited resources available in the most effective manner. Private insurance companies and government run health care services need to assess the appropriateness of a given medical procedure for a particular diagnoses. Determining the legitimacy of a medical procedure thus has become critical in the successful operation of a health care system.

### SUMMARY

Embodiments of the present invention are directed to a computer-implemented method for medical procedure assessment and evaluation. A non-limiting example of the computer-implemented method includes receiving, by a processor, a claim record and storing the claim record as a claim. The method classifies, by the processor, the claim using an intelligent system including an inference engine and a knowledge base and classifies, by the processor, a probability of a diagnostic using the intelligent system. The method calculates, by the processor, using the probability of the diagnostic and the claim a probability of consistency of the diagnostic using the intelligent system and presents, by the processor, the probability of consistency of the diagnostic to a user.

Embodiments of the present invention are directed to a system for medical procedure assessment and evaluation. A non-limiting example of the system includes a memory and a processor coupled to the memory. The processor is operable to execute instructions stored in the memory. The instructions cause the processor to receive a claim record and store the claim record as a claim. The instructions also cause the processor to classify the claim using an intelligent system including an inference engine and a knowledge base and classify a probability of a diagnostic using the intelligent system. The instructions cause the processor to calculate using the probability of diagnostic and the claim a probability of consistency of the diagnostic using the intelligent system and present the probability of consistency of the diagnostic to a user.

Embodiments of the invention are directed to a computer program product for medical procedure assessment and evaluation. The computer program product includes a computer readable storage medium having program instructions embodied therewith. The program instructions are executable by a processor to cause the processor to perform a method. A non-limiting example of the method includes receiving, by a processor, a claim record and storing the claim record as a claim. The method classifies, by the processor, the claim using an intelligent system including an inference engine and a knowledge base and classifies, by the processor, a probability of a diagnostic using the intelligent system. The method calculates, by the processor, using the probability of the diagnostic and the claim a probability of consistency of the diagnostic using the intelligent system and presents, by the processor, the probability of consistency of the diagnostic to a user.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a cloud computing environment according to an embodiment of the present invention;

FIG. 2 depicts abstraction model layers according to an embodiment of the present invention; and

FIG. 3 depicts a flowchart for assessing a request for a procedure by a health provider according to embodiments of the invention; and

FIG. 4 depicts details of an exemplary computing system capable of implementing aspects of the invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

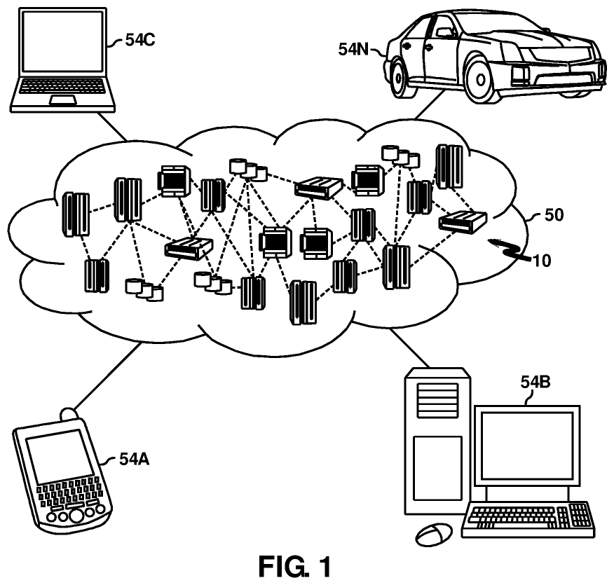

Referring now to FIG. 1, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

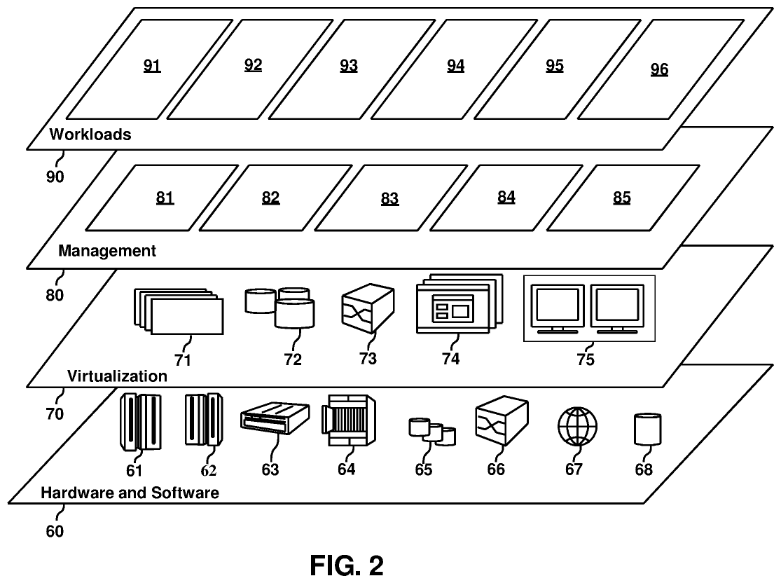

Referring now to FIG. 2, a set of functional abstraction layers provided by cloud computing environment 50 (FIG. 1) is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and medical procedure evaluation and assessment processing 96.

Turning now to an overview of technologies that are more specifically relevant to aspects of the invention, “health interoperability” is the ability of different technology systems and software applications to communicate, exchange data, and use any information that is exchanged. “Terminology” is the ability to represent concepts in an unambiguous manner between a sender and receiver of information. Communications between health information systems relies on structured vocabularies, terminologies, code sets and classification systems that represent health concepts.

Claim data within a medical claim has several limitations, but is a highly valuable data source because it is cheaply obtained and provides information about patient procedures and basic patient characteristics, for example, age and gender. Currently, healthcare plans with insurance companies have limited and labor-intensive procedures to grant permission to patients to execute medical exams. Cheap and common exams, e.g., hemograms, are often automatically allowed because the cost of manual cross-checking with a diagnostic hypothesis is more expensive than the exam itself. More expensive exams, e.g., PET scans, require a healthcare plan doctor to verify if the requested exam is really required and if it is related with a diagnostics hypothesis and patient profile. The current method has several issues. It does not control costs of a large volume of low-cost exams. In addition, it limits the quality and availability that the healthcare provider might give to the patient for more expensive exams, where it may take several hours, or even days, to grant permission.

In order to evaluate and approve the payment, it is necessary to verify if the clinical condition of the patient is compatible with the procedure done, but in the claim one does not have the clinical condition of the patient. In some cases one has the disease in a code if one is fortunate enough to not have this field be empty. To have the clinical condition it is necessary for the provider to have access to the patient's electronic medical record. But access is not enough, clinical conditions have to be interpreted because there are several types of health terminology, and there are cases in which the provider uses none of them.

Another form of evaluation is by the disease that may or may not appear in the claim and that also has several terminologies, as well as by the procedure. In addition, new procedures arise all the time, e.g., in Brazil the terminology Private Health Insurance Unified Terminology (“TUSS”) is modified every three months, and thus having a solution based on a table or list of diseases by procedure becomes quickly obsolete even if done according to existing terminologies.

Turning now to an overview of the aspects of the invention, one or more embodiments of the invention address the above-described shortcomings of the prior art by providing a computer-implemented method to assess if a medical procedure indicated for a patient is consistent with an informed diagnostic hypothesis. The computer-implemented method also defines the probability that an indicated medical procedure for a patient is consistent when a diagnostic hypothesis is not provided.

The above-described aspects of the invention address the shortcomings of the prior art by using a cognitive solution to automatically assess the consistent probability of a procedure indicated for a patient with or without the presence of an informed diagnostic hypothesis. The computer-implemented method combines the knowledge that one has in terminology of procedure and disease with epidemiological disease information to create a method and an intelligent system to classify a given procedure and diagnostic hypothesis and calculate the probability of the consistency of the procedure indicated for the patient.

If the diagnostic hypothesis is not informed, the method combines the knowledge that one has in terminology of procedure and diseases with epidemiological disease information to create a method and an intelligent system to calculate the probability of the diagnostics for the procedure informed. The computer-implemented method provides an automated artificial intelligence system for healthcare plans and insurance companies that is capable of checking requested exams, while granting “real-time” access to exams by checking the consistency of the diagnostics hypothesis and patient profile.

Turning now to a more detailed description of aspects of the present invention, an intelligent system is initially developed and consistently updated when there is an update to terminology or epidemiological information of diseases. The artificial intelligence system includes an inference engine and a knowledge base. The inference engine uses machine learning, ontology, knowledge graphs, rules, and case-based reasoning. The inference engine machine learning may include a neural network, for example. In the initial training of the intelligent system, procedure terminology, disease terminology, and an epidemiological database are supplied to the intelligent system.

Procedure terminology includes, for example, the nature, type, and anatomical region of the procedure. The disease terminology includes the anatomical region that is affected, for example. The epidemiological information of the disease includes age, sex, weather, and seasonality, for example. The intelligent system takes this information and develops the inference engine and knowledge base with the combination of the attributes of the procedure terminology, diagnostic terminology, and epidemiological information. Thus, the intelligent system is created and maintained.

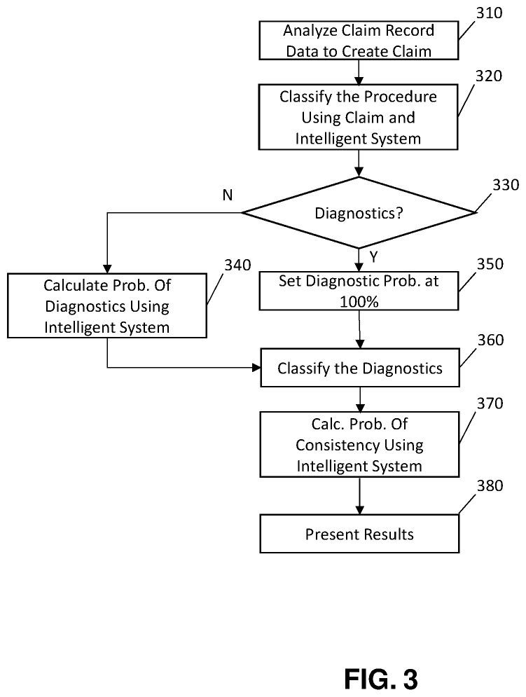

FIG. 3 depicts a flowchart for assessing a request for a procedure by a health provider according to embodiments of the invention. Initially claim record data is received from, for example, an insurance company database and analyzed to create a claim. (stage 310). A procedure stored in the claim is classified using the intelligent system. (stage 320). In particular, a code for the procedure, an anatomical region, a nature of the procedure, and a specialty, e.g. general, clinical, surgery, or diagnostic, for example, may be used in conjunction with the intelligent system to classify the procedure.

A check is made to determine if the field for diagnostics has been completed in the claim or has been left blank. (stage 330). If the field has been left blank, machine learning within the intelligent system is used to calculate the probability of various diagnostics. (stage 340). If the field has been completed, the probability of the diagnositic extracted from the field is set at a value of 100%. (stage 350). After either stage 330 or stage 340, the diagnostic is classified using the extracted or calculated diagnostic and the intelligent system. (stage 360).

The probability of the consistency of the diagnostic is calculated using the intelligent system which is provided with the diagnostic and diagnostic probability and data from the claim, such as age, race, and gender, for example. The result, i.e., the probability of the consistency of the diagnostic, is provided to a user. (stage 380). In the alternative, the probability of the consistency of the diagnostic may be used by a computer system to accept or reject a request to perform or to pay for a procedure.

As an example, when an insurance company approves claims, it wants to know if the procedure is consistent with the patient diagnostic hypothesis in the claims of the provider. So, it uses the computer-implemented method to analyze the claims that it has and separate them into approved claims and those not approved. For the claims that are not approved, it can ask a justification from the provider, refer the claim to an expert for further analysis, and/or inform the provider that it will not pay for reasons of non-compliance.

The insurance company can use the system at the start of the request for the procedure. At the end of the request, the system verifies that the procedure is consistent with the diagnosis, and, if it is correct, send the request to the insurance company for processing. Otherwise, the system requests a justification from the health professional. The insurance company upon receiving the request with the justification sends this information to its analysts that approve or deny the request.

The method and system may be used to alert the health care provider that the payment on request may be denied. Before sending the claim to the insurance company, the provider analyzes it and makes any necessary adjustments, so that it goes without error to the insurance company.

The computer-implemented method can be used to assess a medical student's knowledge. A clinical case with the diagnosis is given, so that the student puts the procedure that she would want be done with the patient. The system displays the hit probability to the student, so that a teacher can assess the student's knowledge. The system can be used for additional types of student learning. The student can make a diagnosis, and the system presents the possible procedures. The student inserts a clinical case with the procedures, and the system presents the possible diagnoses.

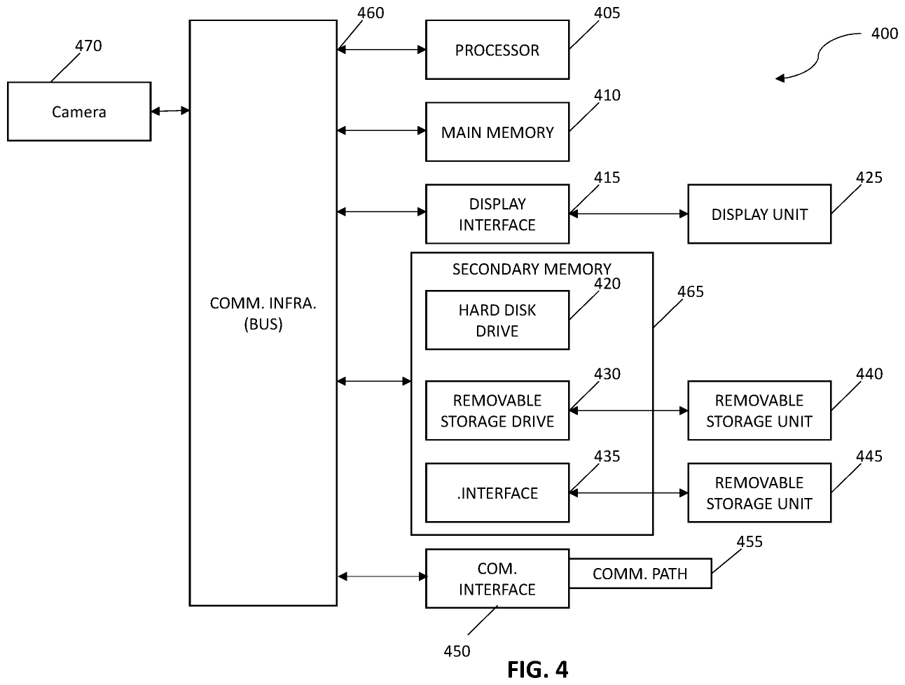

FIG. 4 depicts details of an exemplary computing system capable of implementing aspects of the invention. FIG. 4 depicts a high level block diagram computer system 400, which can be used to implement one or more aspects of the present invention. Computer system 400 may act as a media device and implement the totality of the invention or it may act in concert with other computers and cloud-based systems to implement the invention. More specifically, computer system 400 can be used to implement some hardware components of embodiments of the present invention. Although one exemplary computer system 400 is shown, computer system 400 includes a communication path 455, which connects computer system 400 to additional systems (not depicted) and can include one or more wide area networks (WANs) and/or local area networks (LANs) such as the Internet, intranet(s), and/or wireless communication network(s). Computer system 400 and additional system are in communication via communication path 455, e.g., to communicate data between them.

Computer system 400 includes one or more processors, such as processor 405. Processor 405 is connected to a communication infrastructure 460 (e.g., a communications bus, cross-over bar, or network). Computer system 400 can include a display interface 415 that forwards graphics, text, and other data from communication infrastructure 460 (or from a frame buffer not shown) for display on a display unit 425. Computer system 400 also includes a main memory 410, preferably random access memory (RAM), and can also include a secondary memory 465. Secondary memory 465 can include, for example, a hard disk drive 420 and/or a removable storage drive 430, representing, for example, a floppy disk drive, a magnetic tape drive, or an optical disk drive. Removable storage drive 430 reads from and/or writes to a removable storage unit 440 in a manner well known to those having ordinary skill in the art. Removable storage unit 440 represents, for example, a floppy disk, a compact disc, a magnetic tape, or an optical disk, etc. which is read by and written to by removable storage drive 430. As will be appreciated, removable storage unit 440 includes a computer readable medium having stored therein computer software and/or data.

In alternative embodiments, secondary memory 465 can include other similar means for allowing computer programs or other instructions to be loaded into the computer system. Such means can include, for example, a removable storage unit 445 and an interface 435. Examples of such means can include a program package and package interface (such as that found in video game devices), a removable memory chip (such as an EPROM, or PROM) and associated socket, and other removable storage units 445 and interfaces 435 which allow software and data to be transferred from the removable storage unit 445 to computer system 400. In addition, a camera 470 is in communication with processor 405, main memory 410, and other peripherals and storage through communications interface 460.

Computer system 400 can also include a communications interface 450. Communications interface 450 allows software and data to be transferred between the computer system and external devices. Examples of communications interface 450 can include a modem, a network interface (such as an Ethernet card), a communications port, or a PCM-CIA slot and card, etcetera. Software and data transferred via communications interface 450 are in the form of signals which can be, for example, electronic, electromagnetic, optical, or other signals capable of being received by communications interface 450. These signals are provided to communications interface 450 via communication path (i.e., channel) 455. Communication path 455 carries signals and can be implemented using wire or cable, fiber optics, a phone line, a cellular phone link, an RF link, and/or other communications channels.

In the present description, the terms “computer program medium,” “computer usable medium,” and “computer readable medium” are used to generally refer to media such as main memory 410 and secondary memory 465, removable storage drive 430, and a hard disk installed in hard disk drive 420. Computer programs (also called computer control logic) are stored in main memory 410 and/or secondary memory 465. Computer programs can also be received via communications interface 450. Such computer programs, when run, enable the computer system to perform the features of the present invention as discussed herein. In particular, the computer programs, when run, enable processor 405 to perform the features of the computer system. Accordingly, such computer programs represent controllers of the computer system.

Many of the functional units described in this specification have been labeled as modules. Embodiments of the present invention apply to a wide variety of module implementations. For example, a module can be implemented as a hardware circuit comprising custom VLSI circuits or gate arrays, off-the-shelf semiconductors such as logic chips, transistors, or other discrete components. A module can also be implemented in programmable hardware devices such as field programmable gate arrays, programmable array logic, programmable logic devices or the like.

Modules can also be implemented in software for execution by various types of processors. An identified module of executable code can, for instance, include one or more physical or logical blocks of computer instructions which can, for instance, be organized as an object, procedure, or function. Nevertheless, the executables of an identified module need not be physically located together, but can include disparate instructions stored in different locations which, when joined logically together, comprise the module and achieve the stated purpose for the module.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for medical procedure evaluation and assessment comprising:
receiving, by a processor, a claim record and storing the claim record as a claim;
classifying, by the processor, the claim using an intelligent system including an inference engine and a knowledge base;
classifying, by the processor, a probability of a diagnostic using the intelligent system;
calculating, by the processor, using the probability of the diagnostic and the claim a probability of consistency of the diagnostic using the intelligent system; and
presenting, by the processor, the probability of consistency of the diagnostic to a user.

2. The computer-implemented method of claim 1 further comprising training, by the processor, the intelligent system prior to receiving the claim record.

3. The computer-implemented method of claim 2, wherein training the intelligent system comprises providing the intelligent system data representing procedure terminology, disease terminology, and epidemiological data.

4. The computer-implemented method of claim 1, wherein the diagnostic is provided in the claim.

5. The computer-implemented method of claim 1, wherein the diagnostic is calculated, by the processor, using the intelligent system.

6. The computer-implemented method of claim 1 further comprising approving the claim, by the processor, based on the probability of consistency of the diagnostic.

7. The computer-implemented method of claim 1 further comprising paying the claim, by the processor, based on the probability of consistency of the diagnostic.

8. A system for medical procedure evaluation and assessment comprising:
a memory; and
a processor coupled to the memory, the processor operable to execute instructions stored in the memory, the instructions causing the processor to:
receive a claim record and store the claim record as a claim;
classify the claim using an intelligent system including an inference engine and a knowledge base;
classify a probability of a diagnostic using the intelligent system;
calculate using the probability of the diagnostic and the claim a probability of consistency of the diagnostic using the intelligent system; and
present the probability of consistency of the diagnostic to a user.

9. The system of claim 8, wherein the processor further trains the intelligent system prior to receiving the claim record.

10. The system of claim 9, wherein training the intelligent system comprises providing the intelligent system data representing procedure terminology, disease terminology, and epidemiological data.

11. The system of claim 8, wherein the diagnostic is provided in the claim.

12. The system of claim 8, wherein the diagnostic is calculated, by the processor, using the intelligent system.

13. The system of claim 8, wherein the processor further approves the claim based on the probability of consistency of the diagnostic.

14. The system of claim 8, wherein the processor further pays the claim based on the probability of consistency of the diagnostic.

15. A computer program product for for medical procedure evaluation and assessment, the computer program product comprising a computer readable storage medium having program instructions embodied therewith, the program instructions executable by a computer, to cause the computer to perform a method comprising:
receiving, by a processor of the computer, a claim record and storing the claim record as a claim;
classifying, by the processor, the claim using an intelligent system including an inference engine and a knowledge base;
classifying, by the processor, a probability of a diagnostic using the intelligent system;
calculating, by the processor, using the probability of the diagnostic and the claim a probability of consistency of the diagnostic using the intelligent system; and
presenting, by the processor, the probability of consistency of the diagnostic to a user.

16. The computer program product of claim 15, further comprising training, by the processor, the intelligent system prior to receiving the claim record.

17. The computer program product of claim 15, wherein training the intelligent system comprises providing the intelligent system data representing procedure terminology, disease terminology, and epidemiological data.

18. The computer program product of claim 15, wherein the diagnostic is provided in the claim.

19. The computer program product of claim 15, wherein the diagnostic is calculated, by the processor, using the intelligent system.

20. The computer program product of claim 15 further comprising approving the claim, by the processor, based on the probability of consistency of the diagnostic.
